(19) United States
(12) Patent Application Publication
(10) Pub. No.: US 2018/0345011 A1
(43) Pub. Date: Dec. 6, 2018

(54) DISPOSABLE GASTROINTESTINAL IMPLANTABLE STIMULATOR

(71) Applicant: THE REGENTS OF THE UNIVERSITY OF CALIFORNIA, Oakland, CA (US)

(72) Inventors: Yi-Kai Lo, Los Angeles, CA (US); Wentai Liu, Los Angeles, CA (US)

(73) Assignee: THE REGENTS OF THE UNIVERSITY OF CALIFORNIA, Oakland, CA (US)

(21) Appl. No.: 15/990,753

(22) Filed: May 28, 2018

Related U.S. Application Data
(63) Continuation of application No. PCT/US2016/063886, filed on Nov. 28, 2016.
(60) Provisional application No. 62/260,624, filed on Nov. 29, 2015.

(51) Int. Cl. A61N 1/36; A61N 1/378

(52) U.S. Cl. CPC A61N 1/36007; A61N 1/36053; A61N 1/375; A61N 1/36153; A61N 1/36171; A61N 1/3787

(57) ABSTRACT

A disposable implant that may be positioned inside the gastrointestinal (GI) tract through laparotomy or laparoscopic surgery. The implant may be secured in place using a biodegradable glue or biodegradable suture and is naturally expelled from the body with bowel movement after a certain period of time. In one embodiment, GI implant comprises a coil that receives power from, and sends the recorded physiological information to, an external device through wireless inductive coupling.

### CROSS-REFERENCE TO RELATED APPLICATIONS

This application is a 35 U.S.C. § 111(a) continuation of PCT international application number PCT/US2016/063886 filed on Nov. 28, 2016, incorporated herein by reference in its entirety, which claims priority to, and the benefit of, U.S. provisional patent application Ser. No. 62/260,624 filed on Nov. 29, 2015, incorporated herein by reference in its entirety. Priority is claimed to each of the foregoing applications.

The above-referenced PCT international application was published as PCT International Publication No. WO 2017/091828 on Jun. 1, 2017, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This description pertains generally to medical implants, and more particularly to medical implants for treating post operative ileus.

### 2. Background Discussion

Post-operative ileus (POI) leads to the inflammation of the bowel wall that occurs following abdominal surgery and its economic impact is estimated to be between $¾ billion and $1 billion per year in the United States. Patients with POI manifest abdominal pain, nausea, vomiting, as well as the inability of coordinated propulsive mobility while the current treatment is restricted to the spontaneous recovery of the patient.

POI is not only limited to patients receiving abdominal surgery. There are patients receiving open-heart surgery also reporting symptoms similar to POI, possibly because the sympathetic and parasympathetic nerves governing the GI tack are affected by the surgery.

### BRIEF SUMMARY

A primary premise of the system and methods disclosed herein is that electrical stimulation in the vagus nerve reduces the level of tumor necrosis factor (TNF), indicating the decrease of inflammation. Thus, embodiments of the present technology are configured to treat POI through electrophysiological intervention by stimulating the bowel wall where the nerve ending of VN is located. For the therapeutic treatment of POI, the device performing stimulation is small and easily/conveniently removable after a course of POI treatment.

Accordingly, one aspect of the present technology is an implant that may be positioned inside the gastrointestinal (GI) tract through laparotomy or laparoscopic surgery. The implant may be secured in place using a biodegradable glue or biodegradable suture (e.g. catgut that dissolves in a few days), and is naturally expelled from the body with bowel movement after a certain period of time. By way of example, and not of limitation, the GI implant comprises a coil that receives power from, and sends the recorded physiological information to, an external device through wireless inductive coupling.

Further aspects of the technology will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 shows a schematic diagram of the gastrointestinal (GI) stimulation system of the present description, with disposable GI implant installed inside the GI tract and external stimulator disposed adjacent a patient's abdominal wall.

FIG. 2 shows a schematic illustration of possible placement locations for the disposable GI implant of FIG. 1 within the GI tract.

FIG. 3 shows a detailed schematic diagram of the disposable GI implant of FIG. 1.

FIG. 4 is a schematic illustration of exemplary stimulation parameters control and chip powering in accordance with a method of the present technology.

FIG. 5 is a schematic illustration of an exemplary modulation scheme for reverse data link in accordance with a method of the present technology.

FIG. 6 shows a schematic diagram of the external control device for use with the gastrointestinal (GI) stimulation system of the present description.

FIG. 7 is a schematic diagram of a processing module for implementation of any of the systems or methods of FIG. 1 through FIG. 6.

### DETAILED DESCRIPTION

The technology that is described herein is based on systems and methods for implementation of a disposable miniaturized implant for treatment of Post-Operative Ileus (POI). The primary function of the implant is to provide electrical stimulation to the part of bowel going through surgery to expedite the healing process while recording the smooth muscle activities simultaneously. Disposability of the implant is a key feature, as patients with POI would be less willing to undergo anther surgery to remove the device.

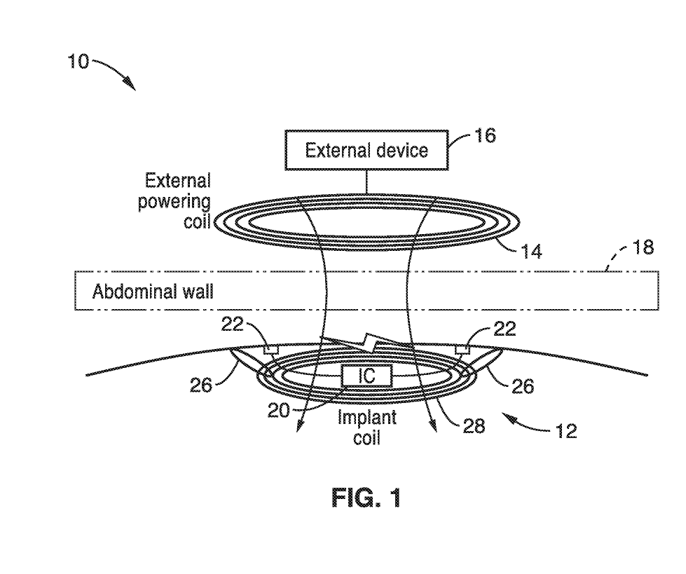

FIG. 1 shows a schematic diagram of the gastrointestinal (GI) stimulation system 10 of the present description, with a disposable GI implant 12 installed inside the GI tract and an external stimulator 16 disposed adjacent a patient's abdominal wall 18.

In a preferred embodiment, the GI implant 12 receives power from, and sends the recorded physiological information to the external device 16, through wireless inductive coupling generated via an implant coil 28 coupled to an IC 20 of implant 12 and external powering coil 14 coupled to the external device 16.

In one embodiment, the implant 12 and the stimulation/recording electrodes 22/24 are adhered on the inner wall of the GI tract by suturing or gluing 26. The suture or glue 26 is configured to be gradually degraded inside the GI tract such that the implant 12 is propelled out of the body with the stool in days or weeks.

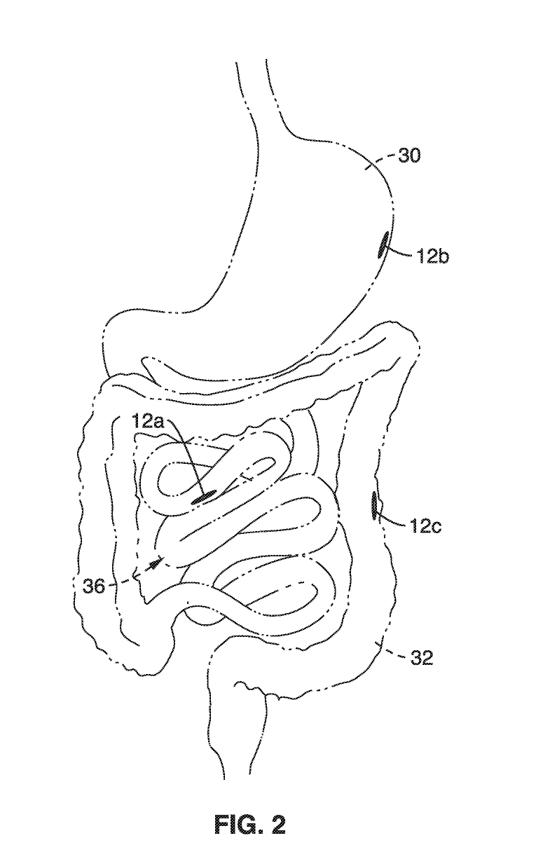

FIG. 2 shows a schematic illustration of possible placement locations for the disposable GI implant 12 within the GI tract. For example, implant locations may comprise one or more of a small intestinal implant 12a with a location of the small intestines 36, stomach implant 12b in the stomach 30, and large intestine implant 12c at a location of the large intestines 32.

The implant 12 can be placed through laparotomy or laparoscopic surgery. The implant 12 is preferably secured in place using a biodegradable glue or biodegradable suture 26 (e.g. catgut that dissolves in a few days). The implant 12 would then be naturally expelled from the body with bowel movement after a certain period of time.

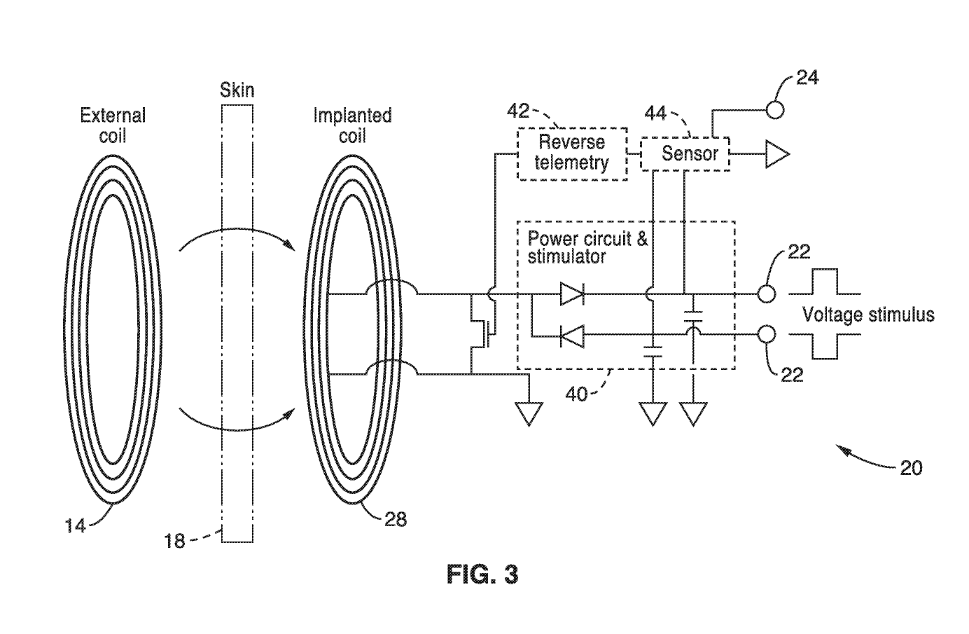

FIG. 3 shows a detailed schematic diagram of the disposable GI implant 12. In the embodiment shown, the implant comprises five primary components as follows: a power circuits/stimulator module 40, a reverse telemetry module 42, and sensor 44, one or more of which may be disposed on IC 20, implantable coil 28, and stimulation/recording electrodes 22/24. As shown in the schematic diagram of FIG. 3, a half-wave rectifier is used to retrieve the induced AC voltage from the implant coil 28 and convert it to DC voltage. The regulated DC voltages are used to perform bipolar electrical stimulation via module power circuit module 40 and electrodes 22.

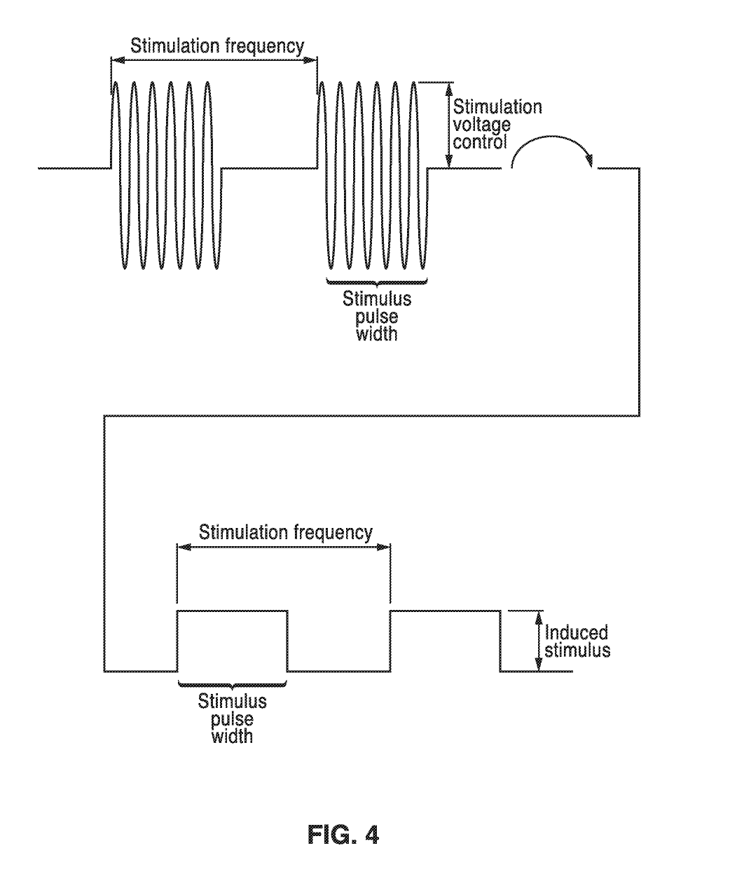

FIG. 4 is a schematic illustration of exemplary stimulation parameters for control and chip powering in accordance with a method of the present technology. Instead of transmitting a continuous power signal to the implant 12, the power signal at the external coil is modulated. Thus, in the implant 12, the stimulator module 40 output produces constant voltage stimulus, whose frequency/pulse width/intensity is determined by its received power signal. The sensor 44 then measures the stimulation voltage and the physiological signal of the smooth muscle via one or more electrodes 24. The reverse telemetry circuits 42 subsequently sends the signal through the same coil 28 using load-shift keying that short the coil when bit 1 is transmitted.

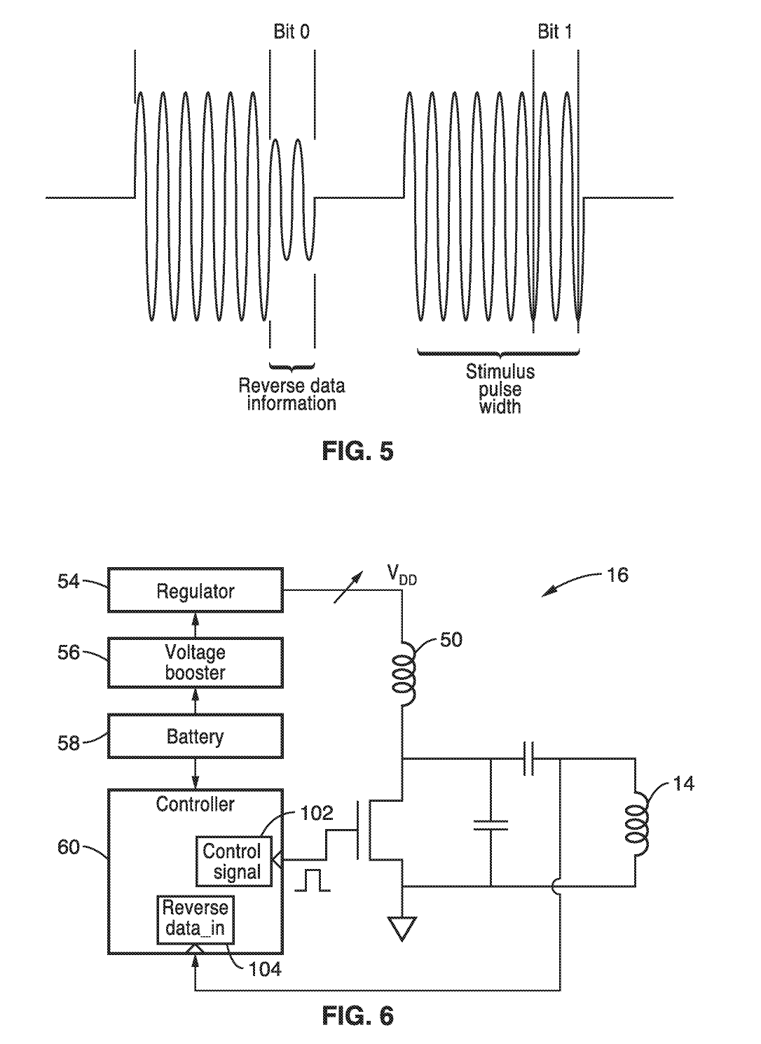

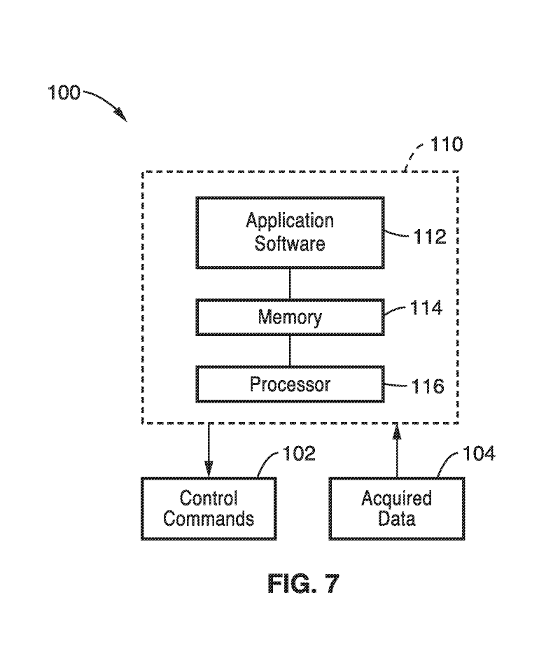

FIG. 5 is a schematic illustration of an exemplary modulation scheme for a reverse data link in accordance with a method of the present technology. The stimulation intensity is mainly determined by the induced voltage at the implanted coil 28 (i.e. amplitude at the primary coil). When transmitting the sensed stimulus intensity and the physiological signal (e.g. acquired data signal 104, FIG. 7) through the power coil 28, the induced voltage is attenuated as its loading condition changes. In one embodiment, the transmitted data 104 is inserted at the very end of each power signal, such that the power signal generated by the primary coil 28 is less influenced. Through the reverse link, a bit 0 can be recognized when there is drop in the amplitude of the induced voltage and vice versa. The length of either bit 1 or bit 0 is varied.

FIG. 6 shows a schematic diagram of the external control device 16 for use with the gastrointestinal (GI) stimulation system 10 of the present description. In the embodiment shown, the external device 16 comprises a power transmitter 50/14 that sends the power signal to the implant 12; a controller 60 that controls the stimulation parameters (i.e. pulse/frequency and intensity); a battery 58; a voltage booster 56 to increase the battery voltage, and a regulator 54 that powers the power transmitter. In the embodiment shown in FIG. 6, a class-E power amplifier is used as an example, but different topology can be employed. The controller 60 senses the information and tunes the stimulation parameters. Note that stimulation intensity is adjusted by varying V_{DD} through the regulator 54.

FIG. 7 is a schematic diagram of a processing module 100 for implementation of any of the systems or methods of FIG. 1 through FIG. 6. Processing module 100 may be implemented for operation of the external device 16, controller 60 of the implant 12, or both. Processing module 100 comprises application programming 112 that may be stored in memory 114 and executable on processor 116 for acquiring sensor data 104 and generating control signals 102. Processing module 100 may comprise a computer 110 or other form of hardware.

Embodiments of the present technology may be described with reference to flowchart illustrations of methods and systems according to embodiments of the technology, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

It will further be appreciated that the terms “programming” or “program executable” as used herein refer to one or more instructions that can be executed by a processor to perform a function as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors. It will further be appreciated that as used herein, that the terms processor, computer processor, central processing unit (CPU), and computer are used synonymously to denote a device capable of executing the instructions and communicating with input/output interfaces and/or peripheral devices.

From the description herein, it will be appreciated that that the present disclosure encompasses multiple embodiments which include, but are not limited to, the following:

1. An implantable apparatus for stimulating tissue, comprising: an implantable coil; a power and stimulator module connected to the implantable coil; a voltage stimulus electrode connected to the power and stimulator module; a reverse telemetry module connected to the implantable coil; a sensor connected to the reverse telemetry module; and a recording electrode connected to the sensor; wherein the implantable coil is configured to couple an external device via a wireless inductive coupling such that the power and stimulator module receives power and commands from the external device to apply a stimulus voltage at a treatment location in a body tissue through the voltage stimulus electrode; wherein the sensor is configured to receive one or more of a stimulus intensity applied by the stimulator module and a physiological signal received from the body tissue.

2. The implantable apparatus of any of the preceding embodiments, wherein the physiological signal is transmitted to the external device through wireless inductive coupling.

3. The implantable apparatus of any of the preceding embodiments: wherein the wireless inductive coupling comprises a modulated power signal; and wherein transmitted data is inserted at the end of the power signal.

4. The implantable apparatus of any of the preceding embodiments: wherein the stimulator module produces a constant voltage stimulus; wherein one or more of the frequency, pulse width, and intensity of the constant voltage stimulus is determined by a received power signal from the external device.

5. The implantable apparatus of any of the preceding embodiments, wherein the reverse telemetry module is configured to transmit the physiological signal through the implanted coil using load-shift keying that shorts the coil when a first bit is transmitted.

6. The implantable apparatus of any of the preceding embodiments, wherein a second bit is recognized as a drop in amplitude of induced voltage.

7. The implantable apparatus of any of the preceding embodiments, wherein the implantable apparatus is configured to be anchored to an internal wall of the body using biodegradable sutures or biodegradable glue.

8. The implantable apparatus of any of the preceding embodiments: wherein the implantable apparatus is configured to be installed at a gastrointestinal treatment location; and wherein the implantable apparatus is configured to be expelled through a bowel after degradation of the biodegradable sutures or biodegradable glue.

9. The implantable apparatus of any of the preceding embodiments, wherein the external device comprises: an external power coil; a power transmitter connected to the external power coil and configured to send a power signal to said implantable coil; and a controller connected to the external power coil and configured to control stimulation parameters and process reverse telemetry of the implantable apparatus.

10. The implantable apparatus of any of the preceding embodiments, wherein the external device further comprises: a battery connected to the controller; a voltage booster connected to the battery; and a regulator connected to the voltage booster and to the power transmitter.

11. A system for stimulating tissue, comprising: (a) an implantable apparatus; (b) man external device; (c) the implantable apparatus comprising: (i) an implantable coil; (ii) a power and stimulator module connected to the implantable coil; (iii) a voltage stimulus electrode connected to the power and stimulator module; (iv) a reverse telemetry module connected to the implantable coil; (v) a sensor connected to the reverse telemetry module; and (vi) a recording electrode connected to the sensor; (d) the external device comprising: (i) an external power coil; (ii) a power transmitter connected to the external power coil and configured to send a power signal to said implantable coil; and (iii) a controller connected to the external power coil and configured to control stimulation parameters and process reverse telemetry of the implantable apparatus.

12. The system of any of the preceding embodiments: wherein the implantable coil is configured to couple to the implantable apparatus via a wireless inductive coupling such that the power and stimulator module receives power and commands from the external device to apply a stimulus voltage at a treatment location in a body tissue through the voltage stimulus electrode; and wherein the sensor is configured to receive one or more of a stimulus intensity applied by the stimulator module and a physiological signal received from the body tissue.

13. The system of any of the preceding embodiments, wherein the physiological signal is transmitted to the external device through wireless inductive coupling.

14. The system of any of the preceding embodiments: wherein the wireless inductive coupling comprises a modulated power signal; and wherein transmitted data is inserted at the end of the power signal.

15. The system of any of the preceding embodiments: wherein the stimulator module produces a constant voltage stimulus; and wherein one or more of the frequency, pulse width, and intensity of the constant voltage stimulus is determined by a received power signal from the external device.

16. The system of any of the preceding embodiments, wherein the reverse telemetry module is configured to transmit the physiological signal through the implanted coil using load-shift keying that shorts the coil when a first bit is transmitted.

17. The system of any of the preceding embodiments, wherein a second bit is recognized as a drop in amplitude of induced voltage.

18. The system of any of the preceding embodiments, wherein the implantable apparatus is configured to be anchored to an internal wall of the body using biodegradable sutures or biodegradable glue.

19. The system of any of the preceding embodiments: wherein the implantable apparatus is configured to be installed at a gastrointestinal treatment location; and wherein the implantable apparatus is configured to be expelled through a bowel after degradation of the biodegradable sutures or biodegradable glue.

20. The system of any of the preceding embodiments, wherein the external device further comprises: a battery connected to the controller; a voltage booster connected to the battery; and a regulator connected to the voltage booster and to the power transmitter.

21. A method for treating post-operative ileus, comprising: installing a disposable implant at a treatment location of a gastrointestinal (GI) tract of a patient; applying an electrical stimulation at the treatment location at or near a vagus nerve ending to reduce a level of tumor necrosis factor (TNF) associate with the GI tract.

22. The method of any of the preceding embodiments: wherein installing a disposable implant comprises anchoring the disposable implant to the treatment location with a biodegradable suture or biodegradable glue; and wherein the disposable implant is configured to be expelled through the GI tract after degradation of the biodegradable sutures or biodegradable glue.

23. The method of any of the preceding embodiments, further comprising: disposing an external stimulator adjacent a patient's abdominal wall; wherein applying an electrical stimulation comprises powering and controlling the disposable implant through an inductive coupling between the external stimulator and disposable implant.

24. The method of any of the preceding embodiments, further comprising: receiving one or more of a stimulus intensity applied by the disposable implant and a physiological signal from a tissue of the GI tract; and transmitting one or more of the stimulus intensity and physiological signal to the external stimulator through the inductive coupling.

25. The method of any of the preceding embodiments: wherein powering and controlling the disposable implant comprises transmitting a modulated power signal to the disposable implant; and wherein transmitted data is inserted at the end of the power signal.

26. The method of any of the preceding embodiments: wherein the modulated power signal comprises a constant voltage stimulus; and wherein one or more of the frequency, pulse width, and intensity of the constant voltage stimulus is determined by a received power signal from the external stimulator.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An implantable apparatus for stimulating tissue, comprising:
an implantable coil;
a power and stimulator module connected to the implantable coil;
a voltage stimulus electrode connected to the power and stimulator module;
a reverse telemetry module connected to the implantable coil;
a sensor connected to the reverse telemetry module; and
a recording electrode connected to the sensor;
wherein the implantable coil is configured to couple an external device via a wireless inductive coupling such that the power and stimulator module receives power and commands from the external device to apply a stimulus voltage at a treatment location in a body tissue through the voltage stimulus electrode; and
wherein the sensor is configured to receive one or more of a stimulus intensity applied by the stimulator module and a physiological signal received from the body tissue.

2. The implantable apparatus of claim 1, wherein the physiological signal is transmitted to the external device through wireless inductive coupling.

3. The implantable apparatus of claim 1:
wherein the wireless inductive coupling comprises a modulated power signal; and
wherein transmitted data is inserted at the end of the power signal.

4. The implantable apparatus of claim 3:
wherein the stimulator module produces a constant voltage stimulus; and
wherein one or more of the frequency, pulse width, and intensity of the constant voltage stimulus is determined by a received power signal from the external device.

5. The implantable apparatus of claim 1, wherein the reverse telemetry module is configured to transmit the physiological signal through the implanted coil using load-shift keying that shorts the coil when a first bit is transmitted.

6. The implantable apparatus of claim 5, wherein a second bit is recognized as a drop in amplitude of induced voltage.

7. The implantable apparatus of claim 1, wherein the implantable apparatus is configured to be anchored to an internal wall of the body using biodegradable sutures or biodegradable glue.

8. The implantable apparatus of claim 5:
wherein the implantable apparatus is configured to be installed at a gastrointestinal treatment location; and
wherein the implantable apparatus is configured to be expelled through a bowel after degradation of the biodegradable sutures or biodegradable glue.

9. The implantable apparatus of claim 1, wherein the external device comprises:
an external power coil;
a power transmitter connected to the external power coil and configured to send a power signal to said implantable coil; and
a controller connected to the external power coil and configured to control stimulation parameters and process reverse telemetry of the implantable apparatus.

10. The implantable apparatus of claim 9, wherein the external device further comprises:
a battery connected to the controller;
a voltage booster connected to the battery; and
a regulator connected to the voltage booster and to the power transmitter.

11. A system for stimulating tissue, comprising:
(a) an implantable apparatus;
(b) an external device;
(c) the implantable apparatus comprising:
(i) an implantable coil;
(ii) a power and stimulator module connected to the implantable coil;
(iii) a voltage stimulus electrode connected to the power and stimulator module;
(iv) a reverse telemetry module connected to the implantable coil;
(v) a sensor connected to the reverse telemetry module; and
(vi) a recording electrode connected to the sensor;
(d) the external device comprising:
(i) an external power coil;
(ii) a power transmitter connected to the external power coil and configured to send a power signal to said implantable coil; and
(iii) a controller connected to the external power coil and configured to control stimulation parameters and process reverse telemetry of the implantable apparatus.

12. The system of claim 11:
wherein the implantable coil is configured to couple to the implantable apparatus via a wireless inductive coupling such that the power and stimulator module receives power and commands from the external device to apply a stimulus voltage at a treatment location in a body tissue through the voltage stimulus electrode; and
wherein the sensor is configured to receive one or more of a stimulus intensity applied by the stimulator module and a physiological signal received from the body tissue.

13. The system of claim 11, wherein the physiological signal is transmitted to the external device through wireless inductive coupling.

14. The system of claim 11:
wherein the wireless inductive coupling comprises a modulated power signal; and
wherein transmitted data is inserted at the end of the power signal.

15. The system of claim 14:
wherein the stimulator module produces a constant voltage stimulus; and
wherein one or more of the frequency, pulse width, and intensity of the constant voltage stimulus is determined by a received power signal from the external device.

16. The system of claim 11, wherein the reverse telemetry module is configured to transmit the physiological signal through the implanted coil using load-shift keying that shorts the coil when a first bit is transmitted.

17. The system of claim 16, wherein a second bit is recognized as a drop in amplitude of induced voltage.

18. The system of claim 11, wherein the implantable apparatus is configured to be anchored to an internal wall of the body using biodegradable sutures or biodegradable glue.

19. The system of claim 16:
wherein the implantable apparatus is configured to be installed at a gastrointestinal treatment location; and
wherein the implantable apparatus is configured to be expelled through a bowel after degradation of the biodegradable sutures or biodegradable glue.

20. The system of claim 11, wherein the external device further comprises:
a battery connected to the controller;
a voltage booster connected to the battery; and
a regulator connected to the voltage booster and to the power transmitter.

21. A method for treating post-operative ileus, comprising:
installing a disposable implant at a treatment location of a gastrointestinal (GI) tract of a patient; and
applying an electrical stimulation at the treatment location at or near a vagus nerve ending to reduce a level of tumor necrosis factor (TNF) associate with the GI tract.

22. The method of claim 21:
wherein installing a disposable implant comprises anchoring the disposable implant to the treatment location with a biodegradable suture or biodegradable glue; and
wherein the disposable implant is configured to be expelled through the GI tract after degradation of the biodegradable sutures or biodegradable glue.

23. The method of claim 21, further comprising:
disposing an external stimulator adjacent a patient's abdominal wall;
wherein applying an electrical stimulation comprises powering and controlling the disposable implant through an inductive coupling between the external stimulator and disposable implant.

24. The method of claim 23, further comprising:
receiving one or more of a stimulus intensity applied by the disposable implant and a physiological signal from a tissue of the GI tract; and
transmitting one or more of the stimulus intensity and physiological signal to the external stimulator through the inductive coupling.

25. The method of claim 23:
wherein powering and controlling the disposable implant comprises transmitting a modulated power signal to the disposable implant; and
wherein transmitted data is inserted at the end of the power signal.

26. The method of claim 25:
wherein the modulated power signal comprises a constant voltage stimulus; and
wherein one or more of the frequency, pulse width, and intensity of the constant voltage stimulus is determined by a received power signal from the external stimulator.
